(19) United States
(12) Patent Application Publication
(10) Pub. No.: US 2010/0311629 A1
(43) Pub. Date: Dec. 9, 2010

(54) METHOD FOR REMOVING WATER-INSOLUBLE SUBSTANCES FROM SUBSTRATE SURFACES

(75) Inventors: Heiko Barg, Speyer (DE); Thomas Subkowski, Ladenburrg (DE); Marvin Karos, Schwetzingen (DE); Claus Bollschweiler, Heidelberg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/680,280

(22) PCT Filed: Sep. 23, 2008
(86) PCT No.: PCT/EP2008/062677

§ 371 Date: Mar. 26, 2010

(30) Foreign Application Priority Data
Sep. 28, 2007 (EP) 07117555.8
Nov. 9, 2007 (EP) 07120415.0

(51) Int. Cl. A61K 8/64; A61Q 1/14; A61Q 19/10

(52) U.S. Cl. 510/136; 510/146

(57) ABSTRACT

The invention relates to a method for removing water-insoluble substances from substrate surfaces by means of a solid dry carrier comprising hydrophobin.

The invention relates to a method for removing water-insoluble substances from substrate surfaces by means of a solid dry carrier comprising hydrophobin.

Methods for removing water-insoluble substances from substrates by means of a carrier from daily life are known in particular from the household. Most of these cleaning or spraying compositions, and also cosmetic compositions for removing makeup or skin cleansing, are based on solutions which comprise organic solvents, for example, alcohol, and emulsifiers or detergents, in order to dissolve the water-insoluble substances on the substrates. In addition, such compositions can also comprise particles, such as scouring agents or peeling preparations, which develop a cleaning effect through mechanical rubbing. The majority of these preparations are applied to the substrate and then wiped off with a cloth, made of textiles, paper or cotton wool, thus cleaning the substrate.

In recent years, moist materials have increasingly been supplied which comprise the corresponding preparations for cleaning the substrates. Particularly in the field of cosmetics, moist cleansing wipes or so-called cotton-wool pads containing the corresponding preparations are known.

The use of hydrophobin in cosmetic preparations is known per se. Hydrophobins are small proteins of about 100 to 150 amino acids, which occur in filamentous fungi, for example Schizophyllum commune. They generally have 8 cysteine units. Hydrophobins can be isolated from natural sources, but can also be obtained by means of genetic engineering methods, as disclosed, for example, by WO 2006/082251 or WO 2006/131564.

US 20030217419 A1 describes the use of the hydrophobin SC3 from Schizophyllum commune for cosmetic preparations for the treatment of therapy materials. Here, cosmetic depots are formed which withstand several washes with shampoo.

The prior art has also proposed the use of hydrophobins for other applications.

WO 96/41882 proposes the use of hydrophobins as emulsifiers, thickeners, surface-active substances, for the hydrophilization of hydrophobic surfaces, for improving the water resistance of hydrophilic substrates, for producing oil-in-water emulsions or water-in-oil emulsions. Furthermore, pharmaceutical applications such as the production of ointments or creams, and also cosmetic applications, e.g. for skin protection or for the production of hair shampoos or hair rinses, are proposed.

EP 1 252 516 discloses the coating of various substrates with a solution comprising hydrophobins at a temperature of from 30 to 80° C. Furthermore, the use as demulsifier (WO 2006/103251), as evaporation retarder (WO 2006/128877) or soiling inhibitor (WO 2006/103215), for example, has been proposed.

It was an object of the present invention to provide a method for removing water-insoluble substances from substrate surfaces which has diverse uses. Moreover, the method was to be gentle for the substrate surface. Additionally, the method was to be simple and permit the removal of the water-insoluble substances with little handling.

Furthermore, it was an object of the invention to provide a novel use for hydrophobin.

The objects are achieved by the method described at the outset and the further subject matters of the present invention. A further subject matter of the present invention relates to cosmetic methods for removing sebum and/or makeup, in which the skin and/or hair surface is treated with a carrier which comprises hydrophobin. Moreover, the use of a carrier which comprises hydrophobin for removing water-insoluble substances is provided by the present invention. Particular embodiments of the invention can be found in the claims, the description and the examples. Furthermore, the invention also comprises combinations of preferred embodiments.

The special feature of the invention lies in the fact that the solid dry carriers loaded with hydrophobin absorb a large amount of water-insoluble substance and at the same time release the absorbed substance again in only small amounts.

In the method according to the invention, solid dry carriers are used which comprise hydrophobin.

Hydrophobins are surface-active polypeptides. They can be isolated from natural sources, but can also be obtained by means of genetic engineering methods.

In principle, hydrophobins of one type or another are suitable.

“Hydrophobin” or “hydrophobins” can be understood as meaning polypeptides of the general formula (I)

Xₙ—C¹—X₁₋₅₀—C²—X₀₋₅—C³—X₁₋₁₀₀—C⁴—X₁₋₁₀₀—C⁵—X₁₋₅₀—C⁶—X₀₋₅—C⁷—X₁₋₅₀—C⁸—Xₘ (I)

where X can be any of the 20 naturally occurring amino acids (Phe, Leu, Ser, Tyr, Cys, Trp, Pro, His, Gln, Arg, Ile, Met, Thr, Asn, Lys, Val, Ala, Asp, Glu, Gly). Here, the radicals X can each be identical or different. Here, the indices positioned next to X are in each case the number of amino acids in the particular part sequence X, C is cysteine, alanine, serine, glycine, methionine or threonine, where at least four of the radicals designated C are cysteine and the indices n and m independently of one another are natural numbers. In general, neither m nor n are zero, but are generally 1 or more. For example, m and n, independently of one another, are from 1 to 500. Preferably, m and n, independently of one another, are from 15 to 300. The amino acids named by C¹ to C⁸ are preferably cysteines; however, they can also be replaced by other amino acids of similar space filling, preferably by alanine, serine, threonine, methionine or glycine. However, at least four, preferably at least five, particularly preferably at least six and in particular at least seven of positions C¹ to C⁸ should consist of cysteines. Cysteines in the proteins according to the invention can either be present in reduced form or form disulfide bridges with one another. Particular preference is given to the intramolecular formation of C—C bridges, in particular those with at least one, preferably two, particularly preferably three and very particularly preferably four intramolecular disulfide bridges. In the case of the above-described replacement of cysteines by amino acids of similar space filling, those pairs of C positions are advantageously replaced which can form intramolecular disulfide bridges with one another.

If cysteines, serines, alanines, glycines, methionines or threonines are also used in the positions referred to by X, then the number of the individual C positions in the general formulae can change accordingly.

Preference is given to using hydrophobins of the general formula (II)

Xₙ—C¹—X₃₋₂₅—C²—X₀₋₂—C³—X₅₋₅₀—C⁴—X₂₋₃₅—C⁵X₂₋₁₅—C⁶—X₀₋₂—C⁷—X₃₋₃₅—C⁸—Xₘ (II)

for carrying out the present invention, where X and C have the above meaning and the indices positioned next to X have the above meaning, the indices n and m are natural numbers. In general, neither m nor n are zero, but are generally 1 or more. For example, m and n, independently of one another, may be from 1 to 500. Preferably, m and n, independently of one another, are from 15 to 300 and at least six of the radicals designated C are cysteine. Particularly preferably, all of the radicals C are cysteine.

Particular preference is given to using hydrophobins of the general formula (III)

Xₙ—C¹—X₅₋₉—C²—C³—X₁₁₋₃₉—C⁴—X₂₋₂₃—C⁵—X₅₋₉—C⁶—C⁷—X₆₋₁₈—C⁸—Xₘ (III),

where X and C have the above meaning, and the indices positioned alongside X have the above meaning. In particular, the indices n and m are natural numbers from 1 to 200. In general, at least six of the radicals designated C are cysteine. Particularly preferably all of the radicals C are cysteine.

The groups Xₙ and Xₘ may be peptide sequences which are naturally linked with the other constituents of the hydrophobin. However, for one or both groups, these may also be peptide sequences which are naturally not linked with the other constituents of the hydrophobin. These are also to be understood as meaning those groups Xₙ and/or Xₘ in which a peptide sequence occurring naturally in the protein is extended by a peptide sequence not occurring naturally in the protein.

The group Xₙ and/or Xₘ can completely or partially comprise peptide sequences which do not occur naturally in the protein. The peptide sequences not occurring naturally in the protein, of which the group Xₙ and/or Xₘ can partially or completely consist, will also be referred to below as fusion partners. These fusion partners are generally at least 20, preferably at least 35, amino acids long. These may, for example, be sequences of from 20 to 500, preferably from 30 to 400 and particularly preferably from 35 to 100 amino acids.

Fusion partners have been disclosed, for example, in WO 2006/082251, WO 2006/082253 and WO 2006/131564.

The fusion partner can be selected from a large number of proteins. It is possible for just a single fusion partner to be linked with the radical of the polypeptide, or else for a plurality of fusion partners to be linked with the radical of the polypeptide. However, it is also possible, for example, for two fusion partners at the positions Xₙ or Xₘ to be linked with the radical of the polypeptide.

Particularly suitable fusion partners are proteins, which occur naturally in microorganisms, in particular in Escherichia coli or Bacillus subtilis. Examples of such fusion partners are the sequences yaad (SEQ ID NO: 16 in WO 2006/082251), yaae (SEQ ID NO: 18 in WO 2006/082251), ubiquitin and thioredoxin. Of high suitability are also fragments or derivatives of these specified sequences, which comprise only part, for example from 70 to 99%, preferably from 5 to 50%, and particularly preferably from 10 to 40%, of the specified sequences, or in which individual amino acids, or nucleotides are changed compared with the specified sequence, the percentage data referring in each case to the number of amino acids.

In a further preferred embodiment, the hydrophobin has, besides the specified fusion partner, as one of the groups Xₙ or Xₘ or as terminal constituent of such a group, also a so-called affinity domain (affinity tag/affinity tail). Here, these are, in a manner known in principle, anchor groups which can interact with certain complementary groups and can serve for easier work-up and purification of the proteins. Examples of such affinity domains comprise (His)ₖ, (Arg)ₖ, (Asp)ₖ, (Phe)ₖ or (Cys)ₖ groups, where k is in general a natural number from 1 to 10. Preferably, it may be a (His)ₖ group, where k is four to six. Here, the group Xₙ and/or Xₘ can consist exclusively of such an affinity domain or else of amino acids linked naturally or not naturally with the radical of the polypeptide, and such an affinity domain.

In another of the preferred embodiments, hydrophobins can also be modified in their polypeptide sequence, for example, by glycosylation, acetylation or else by chemical crosslinking, for example, with glutardialdehyde.

One biological property of the hydrophobins used is the change in surface properties when the surfaces e.g. those of a carrier are coated with the proteins.

The change in the surface properties can be determined experimentally, for example, by measuring the contact angle of a water drop before and after coating the surface with the proteins and ascertaining the difference in the two measurements.

The contact angle measurement procedure is known in principle to the person skilled in the art. The measurements are carried out at room temperature with a water drop of 5 μl and using glass platelets as substrate. The precise experimental conditions for an example of a suitable method for measuring the contact angle are given in the experimental section. Under the conditions specified therein, the hydrophobins used can enlarge the contact angle. Thus, the hydrophobins can enlarge change the contact angle, for example, by at least 20°, preferably at least 25°, particularly preferably at least 30°; 40°, 45° in particular 50°, in each case compared with the contact angle of an identically sized water drop with the uncoated glass surface.

Particularly preferred hydrophobins for carrying out the present invention are the hydrophobins of the type dewA, rodA, hypA, hypB, sc3, basf1, basf2. These hydrophobins including their sequences are disclosed, for example in WO 2006/82251. Unless stated otherwise, the sequences stated below refer to sequences disclosed in WO 2006/82251. An overview table with the SEQ-ID numbers can be found in WO 2006/82251 on page 20.

Of particular suitability according to the invention are the hydrophobins yaad-Xa-dewA-his (SEQ ID NO: 20), yaad-Xa-rodA-his (SEQ ID NO: 22) or yaad-Xa-basf1-his (SEQ ID NO: 24) with the polypeptide sequences stated in brackets, and also the nucleic acid sequences coding therefor, in particular the sequences according to SEQ ID NO: 19, 21, 23. Particularly preferably yaad-Xa-dewA-his (SEQ ID NO: 20) can be used. Also proteins which arise starting from the polypeptide sequences shown in SEQ ID NO. 20, 22 or 24, through replacement, insertion or deletion of at least one, up to ten, preferably five amino acids, particularly preferably 5% of all amino acids, and which still have at least 50% of the biological property of the starting proteins are particularly preferred embodiments. Here, biological property of the proteins is understood as meaning the change in contact angle already described.

Hydrophobins suitable particularly for carrying out the present invention are hydrophobins derived from yaad-Xa-dewA-his (SEQ ID NO: 20), yaad-Xa-rodA-his (SEQ ID NO: 22) or yaad-Xa-basf1-his (SEQ ID NO: 24) by shortening the yaad fusion partner. Instead of the complete yaad fusion partner (SEQ ID NO: 16) with 294 amino acids, a shortened yaad radical can advantageously be used. However, the shortened radical should comprise at least 20, preferably at least 35, amino acids. For example, a shortened radical with 20 to 293, preferably 25 to 250, particularly preferably 35 to 150 and, for example, 35 to 100 amino acids can be used. One example of such a protein is yaad40-Xa-dewA-his (SEQ ID NO: 26 in PCT/EP2006/064720), which has a yaad radical shortened to 40 amino acids.

A cleavage site between the fusion partner or fusion partners and the radical of the polypeptide can be used to cleave off the fusion partner (for example by BrCN cleavage on methionine, factor Xa cleavage, enterokinase cleavage, thrombin cleavage, TEV cleavage etc.).

The hydrophobins present according to the invention in the carrier or the hydrophobins used for producing cosmetic compositions can be prepared chemically by known methods of peptide synthesis, for example by solid-phase synthesis in accordance with Merrifield.

Naturally occurring hydrophobins can be isolated from natural sources using suitable methods. By way of example, reference may be made to Wosten et. al., Eur. J. Cell Bio. 63, 122-129 (1994) or WO 96/41882.

A genetically engineered production method for hydrophobins from Talaromyces thermophilus, which comprise no fusion partner, is described, for example, by US 2006/0040349.

The preparation of hydrophobins which comprise a fusion partner can preferably take place by genetic engineering methods in which a nucleic acid sequence coding for the fusion partner and a nucleic acid sequence for the radical of the polypeptide, in particular DNA sequence, are combined such that, in a host organism through gene expression of the combined nucleic acid sequence, the desired protein is produced. One such preparation method is disclosed, for example, by WO 2006/082251 or WO 2006/082253. The fusion partners make the preparation of the hydrophobins considerably easier. Hydrophobins which comprise a fusion partner are produced in the genetic engineering methods with considerably better yields than hydrophobins which do not comprise a fusion partner.

The hydrophobins produced by the genetic engineering method from the host organisms can be worked up in a manner known in principle and be purified using known chromatographic methods.

In general, purified hydrophobins are used for carrying out the invention.

In a preferred embodiment, the simplified work-up and purification method disclosed in WO 2006/082253, pages 11/12 can be used.

For this, the fermented cells are firstly separated off from the fermentation broth, disrupted and the cell debris is separated from the inclusion bodies. The latter can advantageously take place by centrifugation. Finally, the inclusion bodies can be disrupted, for example by acids, bases and/or detergents in a manner known in principle, in order to release the hydrophobins. The inclusion bodies with the hydrophobins used according to the invention can generally be completely dissolved using just 0.1 m NaOH within about 1 h.

The resulting solutions can, where necessary, following adjustment of the desired pH, be used without further purification for carrying out this invention. The hydrophobins can, however, also be isolated as a solid from the solutions. Preferably, the isolation can take place by means of a spray-granulation or spray-drying, as described in WO 2006/082253, page 12. The products obtained after the simplified work-up and purification method comprise, besides remains of cell debris, generally about 80 to 90% by weight of proteins. Depending on the fermentation conditions, the amount of hydrophobins is generally from 30 to 80% by weight, with regard to the amount of all proteins.

The isolated products comprising hydrophobins can be stored as solids.

The hydrophobins can be used for carrying out this invention as such or else after cleaving off and separating off the fusion partner. A cleavage is advantageously undertaken following isolation of the inclusion bodies and their dissolution.

According to the invention, the hydrophobin may be present in a solid dry carrier. Additionally, the solid dry carrier can comprise a hydrophobin or else a composition of different hydrophobins e.g. a composition which comprises two or three hydrophobins.

The carrier is solid and dry. Dry here means that the carrier has no or only a small moisture content. This moisture content can be ascertained by determining the residual moisture by means of a gravimetric measurement. For this, firstly the weight of the unloaded carrier is ascertained at room temperature. After loading the carrier, it is correspondingly dried. After drying, the weight of the now loaded carrier is ascertained. The difference between these values gives the residual moisture, which is quoted as a percentage based on the weight of the unloaded carrier (100%). However, it is to be noted here that the substances with which the carrier is loaded (hydrophobin etc.) likewise contribute to the weight of the loaded carrier. The residual moisture of the carrier according to the invention can be up to 25% residual moisture, preferably up to 12% residual moisture, particularly preferably up to 7% and very particularly preferably up to 3% residual moisture. However, the carrier may also have absolutely no detectable residual moisture.

Preferably, the carrier is a material which itself has a surface which is not entirely smooth but irregular. For example, any surface can have depressions. The carrier can also have cavities, cells and/or pores. The carrier can, however, also be made of a fibrous material or a material which comprises fibers. Preferably the carrier is a foam, sponge, nonwoven, paper and/or cotton wool.

In one embodiment, the carrier has a hydrophilic surface.

In one embodiment of the present invention, surfaces are referred to as hydrophilic if they do not repel water. Hydrophilic surfaces generally have contact angles toward water of less than 90°.

In addition, in a particular embodiment, the carrier has a small pore size.

In one embodiment of the present invention, the carrier is foam. Foam is a plastic whose structure is formed by many cells (cavities, pores enclosed by base material). Virtually all plastics are suitable for foaming. In one of the embodiments, preference is given to foams which are suitable for cosmetic uses.

In a particular embodiment of the present invention, the carrier is open-cell foams, in particular based on melamine-formaldehyde resins.

Production methods for foams based on polyurethanes are known, for example, from WO 2005/103107 or WO 2006/008054.

Production methods for foams based on melamine-formaldehyde resins are disclosed, for example, in EP-A 17 672, EP-A 37 470 and WO 01/94436. According thereto, a mixture of a melamine-formaldehyde precondensate dispersed or dissolved in an aqueous medium, a propellant, a dispersant and a hardener is heated, foamed and cured. The heating can be undertaken, for example, with the help of hot air, steam, or microwave irradiation. The concentration of the melamine/formaldehyde precondensate in the mixture is generally from 55 to 85% by weight, preferably from 63 to 80% by weight.

The crude density of the open-cell foam based on melamine-formaldehyde resins is generally in the range from 3 to 100 kg/m³, preferably in the range from 5 to 20 kg/m³. The term “crude density” refers in a manner known in principle to the density of the foam including the pore volume. The cell number is usually in the range from 50 to 300 cells/25 mm. The average pore size is usually in the range from 100 to 250 μm (measurement method: systematic NMR analysis with molecular probes or electron microscopy). The tensile strength is preferably in the range from 100 to 150 kPa and the elongation at break in the range from 8 to 20% (measurement method for tensile strength and elongation at break: Werstoff-Fuhrer Kunststoffe, Hellerich, Harsch, Haenle, 9th edition, Carl Hansen Verlag, 2004 pp. 259-275).

In a further embodiment of the present invention, the carrier is a sponge or sponge-like material.

Sponges are generally to be understood as meaning solid or semi-solid elastic foams which consist of gas-filled, for example polyhedron-shaped cells, which are limited by highly viscous and/or solid cell struts. It is possible to use either naturally occurring sponges, semisynthetic or synthetically produced sponge-like structures. Examples of synthetic sponge-like materials are polyurethanes, polyacrylates, poly(meth)acrylic acid derivatives, homopolymers and copolymers of vinyl acetate. The natural and semisynthetic polymers include, inter alia, cellulose, cellulose ethers or cellulose esters such as cellulose acetate and cellulose acetate phthalate. Examples of natural polymers are polysaccharides such as alginates, tragacanth, xanthan gums, guar gum and salts and derivatives thereof. The use of chitin and of chitin derivatives is possible. In the text below, preference is given to using substances with fiber structure, such as scleroproteins, e.g. collagen, keratin, conchagens, fibroin, elastin and chitin. Moreover, it is also possible to use polysaccharides stably crosslinked with one another.

In a further embodiment of the present invention, the carrier is made of textile, nonwoven, paper and/or cotton wool.

The materials can be woven or knitted or be in the form of a composite. The composites include, according to DIN 61210 T2 (no longer in force), nonwoven, paper, cotton wool and felt, which are all to be used according to the invention. Nonwovens are loose materials made of spun fibers (i.e. fibers with limited length) or filaments (endless fibers), mostly produced from polypropylene, polyester or viscose, which are generally held together by the adhesion intrinsic to the fibers. Here, the individual fibers can have a preferred direction (oriented or cross-laid webs) or be unoriented (random webs). The nonwovens may be mechanically bonded by needle punching, intermeshing or by swirling by means of strong water jets. Adhesively bonded nonwovens are formed by gluing the fibers together with liquid binding agents (e.g. acrylate polymers, SBR/NBR, polyvinyl ester, polyurethane dispersions) or by melting or dissolving so-called binder fibers which have been added to the nonwoven during production. During cohesive bonding, the fiber surfaces are partially dissolved by suitable chemicals and bonded by pressure or fused at elevated temperature [J. Falbe, M. Regnitz: Römpp-Chemie-Lexikon, 9th edition, Thieme-Verlag, Stuttgart (1992)].

In a further embodiment of the present invention, cotton wool refers to a loose fiber mass composed of fiber piles and compacted, the fibers being held together by their natural adhesion. They consist of cotton hairs, wool, tillandsia, cellulose and also quartz, inter alia mineral fibers. Cotton wool is preferably used in the form of cotton wool pads or cotton wool buds or in combinations thereof.

For the purposes of the present invention, textiles are to be understood as meaning textile fibers, textile semi-finished and finished products and articles produced therefrom which comprise textile structures serving technical purposes. Textiles can be made of materials of natural origin, for example cotton, wool or flax, or mixed fabrics, for example with cotton/polyester, cotton/polyamide. Textiles can also consist of polyacrylonitrile, polyamide and in particular polyesters or mixtures of materials of natural origin with polyacrylonitrile, polyamide and in particular polyesters.

Paper is generally a flat material which consists of 60 to 95% of mechanically or chemically digested fibers mostly of vegetable origin which are bonded together in an aqueous suspension and are consolidated with the addition of auxiliaries by dewatering on a sieve to give the sheet form. Paper consists of fibers, auxiliaries and water. Besides vegetable fibers from chemical pulp, mechanical pulp and rags (e.g. cotton, hemp fibers), animal, mineral or synthetic fibers are used. In addition, recycled paper as a secondary fiber represents the most important fiber source in terms of amount for paper-making.

In a particular embodiment, the carrier according to the invention is a paper in the form of wipes, such as a paper handkerchief, cosmetic wipe, washing cloth, paper hand towel, paper cleaning cloth or kitchen wipe.

In a further embodiment, the solid dry carrier is attached to an applicator. This applicator can, for example, be made of plastic, glass, wood, metal or textile. One carrier may be attached to the applicator, or else a plurality of identical carriers, e.g. two or three foams.

In a further embodiment, combinations of the carriers can also be attached to the applicator. The table below gives carriers T1 to T68 which are composed of a combination of material characterized in each case by an “x”.

| | | | | | | Cotton |
|---|---|---|---|---|---|---|
| Carrier | Foams | Sponge | Textile | Nonwoven | Paper | wool |
| T1 | x | x | x | x | x | x |
| T2 | x | x | x | x | x | |
| T3 | x | x | x | x | | x |
| T4 | x | x | x | | x | x |
| T5 | x | x | | x | x | x |
| T6 | x | | x | x | x | x |
| T7 | | x | x | x | x | x |
| T8 | x | x | x | x | | |
| T9 | x | x | x | | x | |
| T10 | x | x | | x | x | |
| T11 | x | | x | x | x | |
| T12 | | x | x | x | x | |
| T13 | x | x | x | | | x |
| T14 | x | x | | x | | x |
| T15 | x | | x | x | | x |
| T16 | | x | x | x | | x |
| T17 | x | x | | | x | x |
| T18 | x | | x | | x | x |
| T19 | | x | x | | x | x |
| T20 | x | | | x | x | x |
| T21 | | x | | x | x | x |
| T22 | | | x | x | x | x |
| T23 | x | x | x | | | |
| T24 | x | x | | x | | |
| T25 | x | x | | | x | |
| T26 | x | x | | | | x |
| T27 | x | | x | x | | |
| T28 | | x | x | x | | |
| T29 | x | | x | | x | |
| T30 | x | | x | | | x |
| T31 | x | | | x | x | |
| T32 | x | | | x | | x |
| T33 | x | | | | x | x |
| T34 | | x | x | x | | |
| T35 | | x | x | | x | |
| T36 | | x | x | | | x |
| T37 | | x | | x | x | |
| T38 | | x | | x | | x |
| T39 | | x | | | x | x |
| T40 | | | x | x | | x |
| T41 | | | x | x | x | |
| T42 | | | x | | x | x |
| T43 | | | | x | x | x |
| T44 | x | x | | | | |
| T45 | x | | x | | | |
| T46 | x | | | x | | |
| T47 | x | | | | x | |
| T48 | x | | | | | x |
| T49 | | x | x | | | |
| T50 | | x | | x | | |
| T51 | | x | | | x | |
| T52 | | x | | | | x |
| T53 | | | x | x | | |
| T54 | | | x | | x | |
| T55 | | | x | | | x |
| T66 | | | | x | x | |
| T67 | | | | | x | x |
| T68 | | | | x | | x |

The particular carrier is loaded with hydrophobin. This may be one hydrophobin or else a mixture of different hydrophobins, e.g. a mixture of two or three different hydrophobins.

In one embodiment of the present invention, the carrier is loaded with a mixture comprising hydrophobin.

In principle, the carrier can be loaded in any way with hydrophobin. The person skilled in the art knows of various methods for the loading.

In one variant, the so-called “immersion method” is used in which the carrier is immersed into an immersion bath or is drawn through a bath.

A second variant constitutes the “spray method”, in which the mixture is sprayed onto the carrier.

Further methods used are so-called stripping methods. In these, the carrier runs, for example, as nonwoven, paper, textile or composite webs, past stripping plates, beams or nozzles which are continuously loaded with the hydrophobin-comprising mixtures.

In one embodiment of the present invention, a mixture which comprises hydrophobin and at least water or an aqueous solvent mixture is used for loading the carrier.

Suitable aqueous solvent mixtures comprise water and one or more water-miscible solvents. The selection of such components is only limited inasmuch as the hydrophobins and the other components have to be soluble in the mixture to an adequate degree. Generally, such mixtures comprise at least 50% by weight, preferably at least 65% by weight and particularly preferably at least 80% by weight, of water. Very particularly preferably, only water is used. The person skilled in the art makes a suitable selection from the water-miscible solvents depending on the desired properties of the mixture. Examples of suitable, water-miscible solvents comprise monoalcohols, such as methanol, ethanol or propanol, higher alcohols, such as ethylene glycol or polyether polyols and ether alcohols such as butyl glycol or methoxypropanol.

Preferably, the mixture used for the treatment has a pH of 4 or above, preferably of 6 or above and particularly preferably of 7 or above. In particular, the pH is in the range from 4 to 11, preferably from 6 to 10, particularly preferably from 7 to 9.5 and very particularly preferably from 7.5 to 9. For example, the pH can be from 7.5 to 8.5 or from 8.5 to 9.

To adjust the pH the mixture preferably comprises a suitable buffer. The person skilled in the art selects a suitable buffer depending on the pH range envisaged for the loading. For example, potassium dihydrogenphosphate buffer, tris(hydroxy-methyl)aminomethane buffer (tris buffer), borax buffer, sodium hydrogencarbonate buffer and sodium hydrogenphosphate buffer are to be mentioned. Preference is given to tris buffer.

The concentration of the buffer in the solution is determined by the person skilled in the art depending on the desired properties of the mixture. The person skilled in the art will generally ensure an adequate buffer capacity to achieve the most uniform loading of the carrier possible. A concentration of from 0.001 mol/l to 1 mol/l, preferably from 0.005 mol/l to 0.1 mol/l and particularly preferably from 0.01 mol/l to 0.05 mol/l has proven useful.

The concentration of the hydrophobins in the mixture is selected by the person skilled in the art depending on the desired properties of the loading. At higher concentrations, more rapid loading can generally be achieved. As a rule, a concentration of from 0.1 μg/ml to 1000 μg/ml, preferably from 1 μg/ml to 500 μg/ml, particularly preferably from 10 μg/ml to 250 μg/ml, very particularly preferably from 30 μg/ml to 200 μg/m′ and for example from 50 to 100 μg/ml, has proven useful.

Moreover, the mixture used can optionally comprise further components. Examples of additional components comprise surfactants. Suitable surfactants are, for example, nonionic surfactants which comprise polyalkoxy groups, in particular polyethylene oxide groups. Examples comprise polyoxyethylene stearates, alkoxylated phenols and the like. Further examples of suitable surfactants comprise polyethylene glycol(20) sorbitan monolaurate (Tween® 20), polyethylene glycol(20) sorbitan monopalmitate (Tween® 40), polyethylene glycol(20) sorbitan monostearate (Tween® 60), polyethylene glycol(20) sorbitan monooleate (Tween® 80), cyclohexyl methyl-β D-maltoside, cyclohexyl ethyl-βD-maltoside, cyclohexyl-n-hexyl-β D-maltoside, n-undecyl-β D-maltoside, n-octyl-β D-maltopyranoside, n-octyl-β D-glucopyranoside, n-octyl-β D-glucopyranoside, n-dodecanoylsucrose. Further surfactants are disclosed for example in WO 2005/68087 page 9, line 10 to page 10, line 2. The concentration of surfactants is generally from 0.001 to 0.5% by weight, preferably from 0.01 to 0.25% by weight and particularly preferably from 0.1 to 0.2% by weight, in each case based on the amount of all components in the preparation.

Furthermore, metal ions, in particular divalent metal ions can also be added to the mixture. Metal ions can contribute to a more uniform loading. Suitable divalent metal ions comprise, for example, alkaline earth metal ions such as Ca²⁺ ions. Such metal ions can preferably be added as salts soluble in the formulation, for example, in the form of chlorides, nitrates or carbonate, acetate, citrate, gluconate, hydroxide, lactate, sulfate, succinate, tartrate. For example, calcium chloride or magnesium chloride can be added. The solubility can optionally also be increased through suitable auxiliaries, for example, complexing agents. If present, the concentration on such metal ions is generally from 0.01 to 10 mmol/l, preferably from 0.1 to 5 mmol/l and particularly preferably from 0.5 to 2 mmol/l.

In one embodiment of the present invention the carriers are loaded, in addition to the mixture comprising hydrophobin, with cosmetics such as gel, foam, spray, an ointment, cream, emulsion, suspension, lotion, milk or paste. These cosmetics can bring about a further positive effect on skin and hair.

The cosmetics can comprise the following constituents:
dyes, e.g. those which are known to the person skilled in the art from cosmetics handbooks
cosmetically and/or dermatologically active ingredients, e.g. coloring active ingredients, skin and hair pigmentation agents, tinting agents, tanning agents, bleaches, keratin-hardening substances, antimicrobial active ingredients, photofilter active ingredients, repellant active ingredients, hyperemic substances, keratolytic and keratoplastic substances, antidandruff active ingredients, antiphlogistics, keratinizing substances, antioxidative active ingredients and active ingredients that act as free-radical scavengers, skin-moisturizing or humectant substances, refatting active ingredients, antierythimatous or antiallergic active ingredients and mixtures thereof;
cosmetic compositions for the care and protection of the skin, nail care compositions for decorative cosmetics, skin cosmetic compositions, e.g. face tonics, face masks and other cosmetic lotions.

The preparations can be obtained by mixing the above-described hydrophobin-comprising mixtures with the desired additional aforementioned components and diluting to the desired concentration. The preparations can of course also be obtained by correspondingly dissolving isolated, solid hydrophobins.

The carrier is in any case treated with a hydrophobin-comprising mixture or a hydrophobin-comprising preparation. In order to ensure uniform loading of the carrier, the carrier should be saturated as completely as possible with the mixture or preparation. The loading can in particular be undertaken by immersing the carrier into the mixture or preparation, spraying with the mixture or preparation or perfusing with the mixture or preparation.

As a rule, a certain contact time is required in order to load the carriers. The person skilled in the art chooses a suitable contact time depending on the desired result. Examples of typical contact times are in a period from 0.1 to 12 h, without any intention to limit the invention thereto. It can be influenced, for example, by applying pressure or vacuum.

As a rule, the contact time is dependent on the temperature and on the concentration of the hydrophobin in the mixture or preparation. The higher the temperature and the higher the concentration in the course of the loading process, the shorter the contact time may be. The temperature in the course of the loading process can be room temperature or else may be elevated temperatures. For example, the temperatures may be 5, 10, 20, 30, 40, 50, 60, 70, 80, 90, 100, 110, or 120° C. Preference is given to temperatures of from 15 to 120° C., particularly preferably from 20 to 100° C., and, for example, from 40 to 100° C. or from 70 to 90° C. The temperature can, for example, be introduced by heating the bath in which the carrier to be loaded is immersed. It is also possible, however, to subsequently heat an immersed carrier, for example, with the help of IR rays.

In a preferred embodiment of the invention, the loading with hydrophobin takes place in the presence of microwave irradiation. As a result the contact time can be very considerably reduced. Depending on the energy input, just a few seconds suffice in certain circumstances to load the carrier with hydrophobin.

After the loading, the solvent is removed from the carrier. Preferably, the solvent can be removed mechanically from the carrier by exerting pressure. Optionally, the carrier can be washed beforehand also with water or a preferably aqueous solvent mixture. Removal of the solvent can take place, for example, by simple evaporation in air. Removal of the solvent can, however, also be made easier by increasing the temperature and/or with suitable gas streams and/or applying a vacuum. The evaporation can be made easier by, for example, heating loaded carriers in a drying cabinet or blowing a heated gas stream onto them. The methods can also be combined, for example by drying in a convection drying oven or a drying tunnel. Furthermore, the loaded carrier can also be heated by means of radiation, in particular IR radiation, to remove the solvent. For this, all types of broadband IR emitters, for example NIR, MIR or NIR emitters, can be used. However, it is also possible, for example, to use IR lasers. Such radiation sources are commercially available in diverse radiation geometries.

The temperature and the drying time in the course of drying are set by the person skilled in the art. In general, a drying temperature of from 30 to 130° C., preferably from 50 to 120° C., particularly preferably from 70 to 110° C., very particularly preferably from 75 to 105° C. and for example from 85 to 100° C. has proven useful. What is intended here is the temperature of the carrier itself. The temperature in a drier may of course also be higher. Of course, the higher the drying temperature the shorter the drying time. Likewise, the drying temperatures may be lower and the drying time shorter if the drying is carried out at subatmospheric pressure. The drying time is also governed by the desired residual moisture content of the carrier and can influence this in a decisive manner.

The temperature treatment in the course of loading the carrier and the drying can advantageously be combined with one another. Thus, for example, a carrier firstly can be treated with the mixture or preparation at room temperature and then be dried and heat-treated at elevated temperatures. In a preferred embodiment of the method, elevated temperature is applied at least in one of the two steps “loading” or “drying”. Preferably, a temperature higher than room temperature is applied in both steps.

The loading can be carried out directly after the production process of the carrier, for example by the manufacturer of the carrier himself. However, it can of course also not be carried out until a later time, for example by a further processor or following delivery of the carrier to the end user by him himself.

In a second embodiment of the invention, the loaded carriers can be obtained by carrying out the production of the carrier in the presence of hydrophobin.

In a further embodiment of the invention, the loaded foams can be obtained by carrying out the production of the foam in the presence of hydrophobin.

In the case of the production of open-cell foams based on melamine-formaldehyde condensation products, for this purpose the hydrophobin and optionally further of the aforementioned constituents can be mixed with the aforementioned aqueous solution or dispersion of the melamine-formaldehyde precondensate, a propellant, a dispersant and a hardener. The mixture can then be heated, foamed and cured in a manner known in principle.

It is of particular advantage for the carriers according to the invention and also for the method according to the invention that the hydrophobin also remains stable in the dry state on the carrier.

The solid dry carriers according to the invention are preferably used in a method for removing water-insoluble substances.

As a rule, these are a plurality of substances, although there may also only be one substance.

The solubility in water of the substances which are removed according to the invention from substrate surfaces can range from essentially water-insoluble to completely water-insoluble. In general, the solubility of these substances in water is about 1 g/l at room temperature. Moreover, the solubility in water at room temperature can, however, be lower than 1 g/l.

In one embodiment of the present invention, the water-insoluble substances are fats, oils, waxes and/or compositions comprising fats, waxes and/or oils. Apart from oils, fats and/or waxes, compositions can also comprise further substances.

In one embodiment, the compositions can also comprise a combination of oils, fats and waxes:
a) oils, waxes and fats
b) oils and waxes
c) oils and fats
d) waxes and fats

In one embodiment of the present invention, oils are water-insoluble, liquid organic compounds with a relatively low vapor pressure. The oils include fatty oils, essential oils, mineral oils and silicone oils.

Fatty oils are fats, i.e. mixtures of fatty acid triglycerides, which are liquid at room temperature, whereas fats are solid at room temperature.

Essential oils are oily, water-vapor-volatile extracts from plants or parts of plants, which have a strong characteristic odor depending on the original plant. They consist for the most part of terpenes (example: lemon oil). Essential oils can, however, also be produced synthetically.

Mineral oils are obtained from crude oils or coals and are hydrocarbon compounds. From a chemical point of view, most of the compounds in the substance mixtures are types of alkanes.

Silicone oils are based on polymers and copolymers of silicon-oxygen units and organic side chains. They are relatively insensitive toward oxidation, heat and other influences.

In one embodiment of the present invention, fats are esters of the trihydric alcohol glycerol (propane-1,2,3-triol) with three, mostly different, predominantly even-numbered and unbranched aliphatic monocarboxylic acids, the so-called fatty acids.

In one embodiment of the present invention, waxes are substances which are nowadays defined by their mechanicophysical properties. Their chemical composition and origin, by contrast, vary greatly. A substance is referred to as a wax if it is kneadable at 20° C., is solid to brittly hard, has a coarse to finely crystalline structure, is translucent to opaque in terms of color, but is not glass-like, melts above 40° C. without decomposition, is readily liquid (a little viscous) a little above the melting point, has a highly temperature-dependent consistency and solubility and it can be polished using light pressure.

Animal and vegetable waxes include the lipids. Main components of these substance mixtures are esters of fatty acids with long-chain, aliphatic, primary alcohols. Myricin is, for example, an ester of palmitic acid with myristyl alcohol and the main constituent of beeswax. These esters differ in their structure from the fats, which are triglycerides of fatty acids.

Animal waxes are, for example, spermaceti and beeswax. Vegetable waxes are, for example, sugar cane wax, carnauba wax of the wax palm. Jojoba oil is not a triglyceride and thus actually not an oil, but in chemical terms is a liquid wax.

Geological earth waxes (ozokerite and the ceresin produced therefrom) consist essentially of hydrocarbons.

Synthetic waxes are primarily obtained from crude oil. The main product is hard paraffin, which is used, for example, for candles or shoe cream. For specific applications, natural waxes are chemically modified or completely synthesized (polyethylenes, copolymers). Soya wax can also be produced from soya by hydrogenation.

In one embodiment of the present invention the water-insoluble substances are sebum and/or makeup.

In one embodiment of the present invention, makeup is to be understood as meaning decorative cosmetics or makeup.

Decorative cosmetics are substances which are applied for single use on (facial) skin, lips, hair or nails, for the exclusive or predominant purpose of changing their appearance within a short time to a significant degree, mostly in terms of color, but reversibly.

In general, sebum is a skin surface fat which, to a lesser extent, consists of the horny fat, a by-product of keratinization, and originates from the sebaceous glands. The average composition of sebum consists of: triglycerides (19.5-49.4% mass content), wax esters (22.6-29.5% mass content), fatty acids (7.9-39.0% mass content), squalene (10.1-13.9% mass content), diglycerides (1.3-4.3% mass content), cholesterol esters (1.5-2.6% mass content), cholesterol (1.2-2.3% mass content).

In one embodiment of the present invention, a carrier comprising hydrophobin is used in a cosmetic method for removing sebum from skin.

In a further embodiment of the present invention, a carrier comprising hydrophobin is used in a cosmetic method for removing sebum from hair. In a further embodiment of the present invention, a carrier comprising hydrophobin is used in a cosmetic method for removing makeup from skin. In a further embodiment of the present invention, a carrier comprising hydrophobin is used in a cosmetic method for removing makeup from hair. In a further embodiment of the present invention a carrier comprising hydrophobin is used in a cosmetic method for removing makeup and sebum from skin. In a further embodiment of the present invention, a carrier comprising hydrophobin is used in a cosmetic method for removing makeup and sebum from hair.

In the method according to the invention water-insoluble substances are removed from substrate surfaces.

The substrates used may be solids. The solids may be amorphous, crystalline or partially crystalline. A substrate can be composed of one or more solids. It is possible that a substrate comprises gas and/or liquid inclusions.

In one embodiment of the present invention, the substrates consist of water or glass, plastic, wood, metal, textile, feathers, fur, skin and/or hair.

Water can include, for example, seas, oceans, rivers, streams, puddles and swimming pools.

Glass can comprise technical glasses, optical glasses and decorative glasses. Examples of these are sheet glasses such as windowpanes and mirrors.

In general, a plastic is a solid body whose base constituent is synthetically or semisynthetically produced polymers with organic groups. Plastics are used in very many fields, such as, for example, in automobile construction, building industry, household and gardens.

In general, wood comprises lignified plant tissue. In one embodiment, wood is also understood as meaning coated wood, as is used, for example, in the manufacture of furniture, parquet and similar floor coverings and toys.

In general, the term metals is understood as meaning all chemical elements apart from hydrogen, carbon, phosphorus, selenium, iodine, helium, neon, argon, krypton, xenon, radon and ununoctium. In addition, the term metal also covers stainless steel, for example, of domestic appliances or cutlery. Stainless steel (according to DIN EN 10020) is a name for alloyed or unalloyed steels with a particular degree of purity, for example, steels whose sulfur and phosphorus content (so-called iron companions) does not exceed 0.025%.

In addition, the term also covers metal alloys.

In one embodiment of the present invention, the term fur relates to animal fur. In one embodiment of the present invention, the term feathers relates to animal feathers.

In one embodiment of the present invention the term skin relates to human facial skin.

The substrate surface is primarily the outer layer of the substrate to be treated. The substrate surface can be smooth, level, not entirely smooth but irregular, for example each surface can have depressions. The substrate surface can also have cavities, cells and/or pores.

The method according to the invention is characterized by the simple and easy handling of the carriers comprising hydrophobin. The solid dry carrier loaded with hydrophobin removes the water-insoluble substances through simple contacting of the substrate surface, e.g. in the form of wiping or dabbing with gentle pressure.

The method according to the invention ensures that water-insoluble substances are removed gently using a carrier.

Apart from being used in the cosmetics sector already described, the present invention can be used in many other fields such as, for example, in the home, environment, transportation and industry.

In industry, for example, the method according to the invention can be used for cleaning industrial plants or sections of plants. This is primarily of great interest in water-free zones or areas which can be thoroughly cleaned using the solid dry carrier according to the invention.

The present invention can also be used, for example, for removing oil contaminations on the land and/or in water. One advantage over conventional methods is that with the methods according to the invention, more oil is absorbed by the carriers loaded with hydrophobin and said oil is released again only in very small amounts. Consequently, firstly only very little oil-contaminated material which has to be disposed of is produced, and secondly the reduced release of the oil prevents further contamination of the environment and of people.

Furthermore, the cleaning of automobiles or parts of automobiles, such as wheel rims, can be undertaken using the present invention. The soilings e.g. on the wheel rim are readily absorbed by the carrier according to the invention, and since said carrier releases the soilings again only in very small amounts, the person doing the cleaning does not come into contact with the removed soilings.

The dry wipes according to the invention with a very low residual moisture are also highly suited for removing soilings (e.g. fingerprints) from smooth surfaces, e.g. plastic surfaces, mirrors or panes of glass. The method according to the invention has the advantage that it is not necessary to clean the entire surface, but instead cleaning can take place partially.

The advantage of the present invention is available in a method for removing water-insoluble substances from substrate surfaces that can be used widely. Moreover, the method is gentle to the substrate surface. Additionally, the method is simple and permits the removal of the water-insoluble substances with little handling.

### EXAMPLE 1

### Removal of Sebum by Means of a Foam Loaded with Hydrophobin

Cube-shaped samples (2.5 cm×2.5 cm×2.5 cm) of an open-cell melamine-formaldehyde foam with a density of 9 kg/m³ (Basotect®, BASF Aktiengesellschaft) were saturated with a solution of 0.1 g/l of hydrophobin A (SEQ ID 20 from WO 2007/14897) or hydrophobin B (SEQ ID 26 from WO 2007/14897). The solution with the saturated foam cube was heated at 60° C. for 15 h. The aqueous solution was then decanted off. The foam cubes were freed from the majority of the absorbed liquid by squeezing, washed several times with ultra pure water and squeezed and dried to constant weight at 40° C.

The removal of natural sebum was tested on untreated human skin, in the forehead area. Small cut cubes (edge length about 2.5 cm) were passed over the skin with gentle pressure.

Determination of the remaining natural sebum on the skin was evaluated using a Sebumeter SM810 from Courage+Khazaka Electronic GmbH. Prior to the measurement, a zero adjustment of the instrument has to be carried out on the film for grease measurement in accordance with the manufacturer's instructions.

Table of Results

| | | Natural sebum |
|---|---|---|
| No. | Sample | remaining (%) |
| K | Control (sponge without hydrophobin) | 100 |
| C1 | Sponge with hydrophobin B | 21 |
| C2 | Sponge with hydrophobin A | 28 |

“K” here signifies the control experiment and “C” the comparative experiment according to the invention.

The loaded foam cubes exhibited improved removal of natural sebum, measured on untreated human skin.

### EXAMPLE 2

### Hydrophobin-Loaded Foam Cubes for Absorbing Synthetic Sebum

For the experiment, 20 g of synthetic sebum (for composition see below) were placed in a watchglass and a foam cube of an open-cell melamine-formaldehyde foam with a density of 9 kg/m³ (Basotect®, BASF Aktiengesellschaft) with an edge length of 15×15 mm was immersed for 1 min. Here, an untreated foam cube, a foam cube loaded with hydrophobin B and a foam cube “loaded” with casein (analogous to the loading of hydrophobin B, see Example 1) were compared.

The experiment was likewise also carried out with pure capric/caprylic triglyceride (Miglyol® 812, Sasol).

The absorbed amount of sebum/triglyceride was ascertained by means of an analytical balance.

The sebum/triglyceride absorption was calculated relative to the weight of the foam cube. The foam cubes were then placed on filter paper (Porringer type 1243/90) for 30 min. The weight was ascertained again using the analytical balance and the sebum transfer was calculated.

Additionally, the cubes were placed on to water and their behavior observed.

### Result:

| | | Sebum | Sebum |
|---|---|---|---|
| No. | Carrier: foam cube | absorption in % | transfer in % |
| C3 | loaded with hydrophobin B | 74.5 | 3 |
| K2 | untreated | 60.5 | 4 |
| K3 | loaded with casein | 39.8 | 5 |

It was found that the carrier according to the invention (C3) was able to absorb the largest amount of synthetic sebum and then transferred the least amount of synthetic sebum. A carrier loaded with an arbitrarily chosen comparison protein (casein) exhibited a clearly reduced sebum absorption, and also a higher subsequent sebum transfer.

### Composition of the Synthetic Sebum:

| | |
|---|---|
| 41.0% | Triglycerides |
| 25.0% | Wax ester |
| 16.0% | Fatty acids |
| 13.0% | Squalene |
| 1.0% | Diglycerides |
| 2.0% | Cholesterol ester |
| 2.0% | Cholesterol |

### EXAMPLE 3

### Loading of Cotton Wool Pads with Hydrophobin B

### Preparation of the Solutions:

Hydrophobin B: hydrophobin B (granules or powder) was firstly predissolved in water at room temperature. The maximum solubility was 50 mg/ml (=5%).

To increase the rate, the solution was heated to 60° C. Following complete dissolution, the protein content was determined by means of Bradford.

Loading buffer: the loading buffer was a 50 mM tris buffer. 1 mM CaCl₂ was added and the pH was adjusted to 8 with 32% HCl.

### Starting the Loading:

The buffer was poured into a beaker and the predissolved hydrophobin B was added until the end concentration was 50 μg/ml and stirred.

The cotton wool pads were placed into the beaker. They were completely immersed into the solution.

### Incubation:

The beaker was covered and then incubated for 16 h in a heating cabinet at 50° C.

### Washing:

The pads were then thoroughly washed under running demineralized water.

### Drying:

The cotton wool pads were dried in a heating cabinet at 50° C. for 24 h.

### Activity:

The success of the loading could then be checked by placing a drop of water (about 50 μl) on the cotton wool pad. If the drop remained on the surface of the pad treated with hydrophobin for longer compared to an untreated pad (about 5 seconds) instead of soaking in, the loaded cotton wool pad was used further.

### EXAMPLE 4

### Hydrophobin-Loaded Cotton Wool Pads for Absorbing Synthetic Sebum

### 1. Experiment:

For the experiment 20 g of synthetic sebum (composition see Example 2) or in an experiment variant capric/caprylic triglyceride (Miglyol® 812, Sasol) were placed in a watchglass, and the untreated and hydrophobin B-loaded cotton wool pads were immersed for 1 min (loading see Example 3).

The cotton wool pads were weighed down with a weight and then placed into a vessel containing water.

### 2. Experiment:

A drop of water was placed onto the untreated and hydrophobin-B-loaded cotton wool pads and it was observed how quickly the drop soaks in (loading see Example 3).

### Results:

For Experiment 1:

The untreated cotton wool pad transferred the sebum/triglyceride rapidly again. The cotton wool pad swelled considerably.

The cotton wool pad treated with hydrophobin B held the sebum/triglyceride considerably better and also remained dimensionally stable.

### For Experiment 2:

In the case of the untreated cotton wool pads, the drop soaked in immediately, whereas the cotton wool pad treated with hydrophobin B held the drop on the surface for significantly longer (bead effect).

### EXAMPLE 5

### Absorption and Inclusion of Oils or Sebum by Means of Foam Coated with Hydrophobin

The experiment below shows that a Basotect sponge coated with hydrophobin exhibits a very good absorption of oils, which also remain in the sponge as a result of the hydrophobin coating. This effect is not based on a surfactant property, as could be assumed, as a comparison with sodium dodecylsulfate shows.

In each case, cube-shaped samples (2.5 cm×2.5 cm×2.5 cm) 5 of an open-cell melamine-formaldehyde foam with a density of 9 kg/m³ (Basotect®, BASF AG) were placed in a glass flask and saturated with a solution of 0.1 g/l of hydrophobin A (SEQ ID 20 from WO2007/14897) or hydrophobin B (SEQ ID 26 from WO2007/14897). Likewise, for comparison, one cube was saturated with a 1% strength SDS solution (sodium dodecylsulfate). The solutions with the saturated foam cubes were heated at 60° C. for 15 h. The aqueous solution was then decanted off.

The foam cubes were freed from the majority of the absorbed liquid by squeezing, washed several times with ultra pure water and squeezed and dried to constant weight at 40° C.

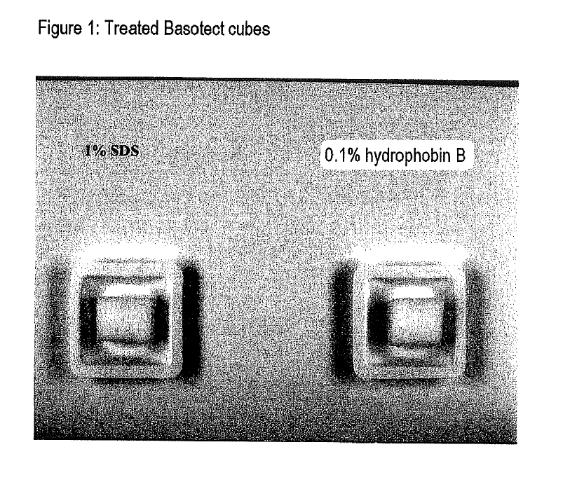

FIGS. 1 to 5:

FIG. 1: Treated Basotect cubes

The cubes were then immersed in cyclomethicone which had been dyed with Sudan Red for better visualization.

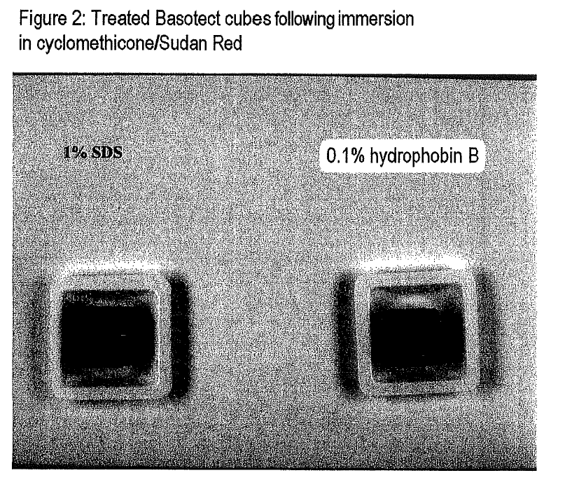

FIG. 2: Treated Basotect cubes following immersion in cyclomethicone/Sudan Red

The cubes were then immersed in water. Here, it can be clearly seen that the Basotect sponge treated with SDS immediately releases the cyclomethicone oil again and the oil is not retained in the sponge. In contrast to this, the cubes coated with hydrophobin A or B barely exhibit any release of the oil.

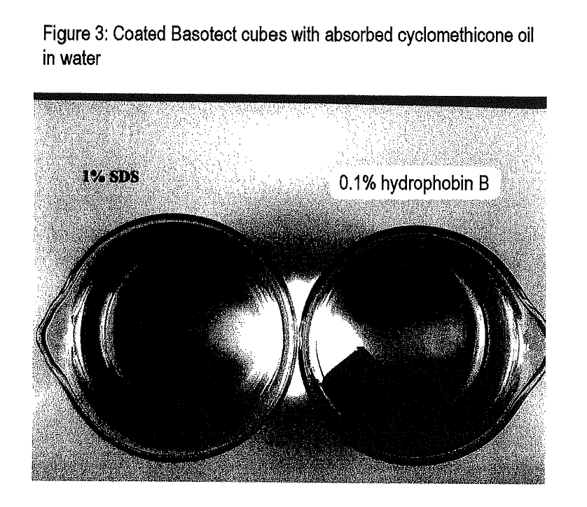

FIG. 3: Coated Basotect cubes with absorbed cyclomethicone oil in water.

Immediately after immersion, bleeding of the SDS-coated sponge can be seen, whereas the hydrophobin-coated sponge does not release any oil.

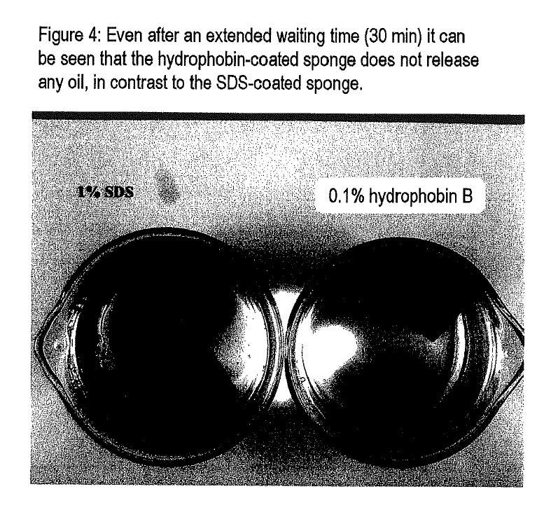

FIG. 4: Even after an extended waiting time (30 min) it can be seen that the hydrophobin-coated sponge does not release any oil, in contrast to the SDS-coated sponge.

Following the immersion procedure, the cubes were carefully sliced using a scalpel. Here, by virtue of the intense red coloration of the hydrophobin treated sponge, it is possible to see that the oil has remained in the sponge, whereas the sponge treated with SDS has led to no recognizable coating of the sponge and has almost completely released the oil again.

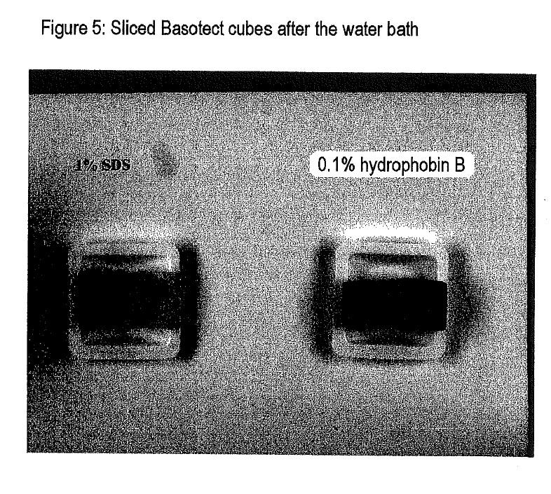

FIG. 5: Sliced Basotect cubes after the water bath. The intense red coloration indicates that the cyclomethicone oil has remained in the hydrophobin coated sponge. By contrast, SDS seemingly leads to no coating and the oil simply escapes from the sponge again.

The same results were achieved both with hydrophobin A and also hydrophobin B. Instead of cyclomethicone, the effect can also be demonstrated with other oils or sebum.

1. A method for removing water-insoluble substances from substrate surfaces by means of a solid dry carrier comprising hydrophobin.

2. The method according to claim 1, wherein the water-insoluble substances are fats, oils, waxes or compositions comprising fats, waxes and/or oils.

3. The method according to claim 1, wherein the water-insoluble substances are sebum and/or makeup.

4. The method according to claim 1, wherein the substrates are: water or glass, plastic, wood, metal, textile, skin and/or hair.

5. The method according to claim 1, wherein the solid dry carrier is made of foam, sponge, textile, nonwoven, paper and/or cotton wool.

6. A cosmetic method for removing sebum and/or makeup, wherein the skin and/or hair surfaces are treated with a solid dry carrier comprising hydrophobin.

7. The use of a solid dry carrier comprising hydrophobin for removing water-insoluble substances from substrate surfaces.

8. The use according to claim 7, wherein the water-insoluble substances are fats, oils, waxes or compositions comprising fats, waxes and/or oils.

9. The use according to claim 7, wherein the water-insoluble substances are sebum and/or makeup.

10. The use according to claim 7, wherein the substrates are water or glass, plastic, wood, metal, textile, skin and/or hair.

11. The use according to claim 7, wherein the solid dry carrier is made of foam, sponge, textile, nonwoven, paper and/or cotton wool.

12. The use of a solid dry carrier comprising hydrophobin in a cosmetic method for removing sebum and/or makeup from skin and/or hair surfaces.
